(19) United States
(12) Patent Application Publication
(10) Pub. No.: US 2018/0070803 A1
(43) Pub. Date: Mar. 15, 2018

(54) IMAGING DEVICE AND ENDOSCOPE SYSTEM

(71) Applicant: OLYMPUS CORPORATION, Tokyo (JP)

(72) Inventor: Masato MIKAMI, Tokyo (JP)

(73) Assignee: OLYMPUS CORPORATION, Tokyo (JP)

(21) Appl. No.: 15/812,211

(22) Filed: Nov. 14, 2017

Related U.S. Application Data
(63) Continuation of application No. PCT/JP2015/065407, filed on May 28, 2015.

(51) Int. Cl. A61B 1/00; A61B 1/05; G02B 23/24

(52) U.S. Cl. CPC A61B 1/00163; A61B 1/05; G02B 23/2407; A61B 1/06; A61B 1/00096; A61B 1/00126; A61B 1/00009; A61B 1/00117

(57) ABSTRACT

An imaging device includes: an optical system configured to concentrate incident light; an image sensor configured to receive the light entering from the optical system and perform photoelectric conversion to generate an electrical signal; a flexible printed board on which a signal cable and an electronic component are mounted, the flexible printed board being connected to an electronic pad of the image sensor; and a light-shielding member arranged between the optical system and the image sensor, a part of the light-shielding member being folded or bent to be in contact with a side surface of the optical system, and the light-shielding member being configured to shield the light entering from a direction of the side surface of the optical system and/or from between the optical system and the image sensor. The light-shielding member is a board including a circuit layer and electrically conducting with the flexible printed board.

### CROSS-REFERENCE TO RELATED APPLICATION

This application is a continuation of PCT international application Ser. No. PCT/JP2015/065407 filed on May 28, 2015 which designates the United States, incorporated herein by reference.

### BACKGROUND

The present disclosure relates to an imaging device and an endoscope system using the imaging device.

In the related art, endoscope devices have been widely used for various examinations in the medical field and the industrial field. Among the endoscope devices, a medical endoscope device performs an observation of a portion to be examined by allowing a long and narrow insertion portion to be inserted into a body cavity of a subject such as a patient, the insertion portion having flexibility and having an imaging device built in a distal end portion. Reduction in the diameter of the insertion portion is required in consideration of easy introduction to the subject.

In such an endoscope device, light from an adjacent illumination system and the like other than an objective optical system needs to be prevented from entering an image sensor. For example, an imaging device for endoscope is disclosed (for example, see JP 11-249030 A), in which an image sensor is provided outside a holding frame that holds an objective optical system, to reduce the diameter, and a light-shielding member is provided around an optical member and the like installed between the image sensor and the objective optical system.

### SUMMARY

An imaging device according to one aspect of the present disclosure may include: an optical system configured to concentrate incident light; an image sensor configured to receive the light entering from the optical system and perform photoelectric conversion to generate an electrical signal; a flexible printed board on which a signal cable and an electronic component are mounted, the flexible printed board being connected to an electronic pad of the image sensor; and a light-shielding member arranged between the optical system and the image sensor, a part of the light-shielding member being folded or bent to be in contact with a side surface of the optical system, and the light-shielding member being configured to shield the light entering from a direction of the side surface of the optical system and/or from between the optical system and the image sensor, wherein the light-shielding member is a board including a circuit layer and electrically conducting with the flexible printed board.

The above and other objects, features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram schematically illustrating an overall configuration of an endoscope system according to a first embodiment;

FIG. 2 is a partial sectional view of a distal end of an endoscope illustrated in FIG. 1;

FIG. 3 is a perspective view of an imaging device used in the endoscope system according to the first embodiment;

FIG. 4 is a sectional view of the imaging device of FIG. 3;

FIG. 5 is a development view of a flexible printed board used in the imaging device of FIG. 3;

FIG. 6 is a perspective view of an imaging device according to a second embodiment;

FIG. 7 is a sectional view of the imaging device of FIG. 6;

FIG. 8 is a development view of a flexible printed board used in the imaging device of FIG. 6;

FIG. 9 is a perspective view of an imaging device according to a third embodiment;

FIG. 10 is a sectional view of the imaging device of FIG. 9; and

FIG. 11 is a development view of a flexible printed board used in the imaging device of FIG. 9.

### DETAILED DESCRIPTION

Hereinafter, an endoscope device including an imaging module will be described as forms for implementing the present disclosure (hereinafter, referred to as “embodiments”). The present disclosure is not limited by the embodiments. Further, the same portion is denoted with the same sign in the illustration of the drawings. Further, it should be noted that the drawings are schematic, and relationship between the thickness and the width of members, ratios of members, and the like are different from reality. Further, portions having different dimensions and ratios are included between the drawings.

### First Embodiment

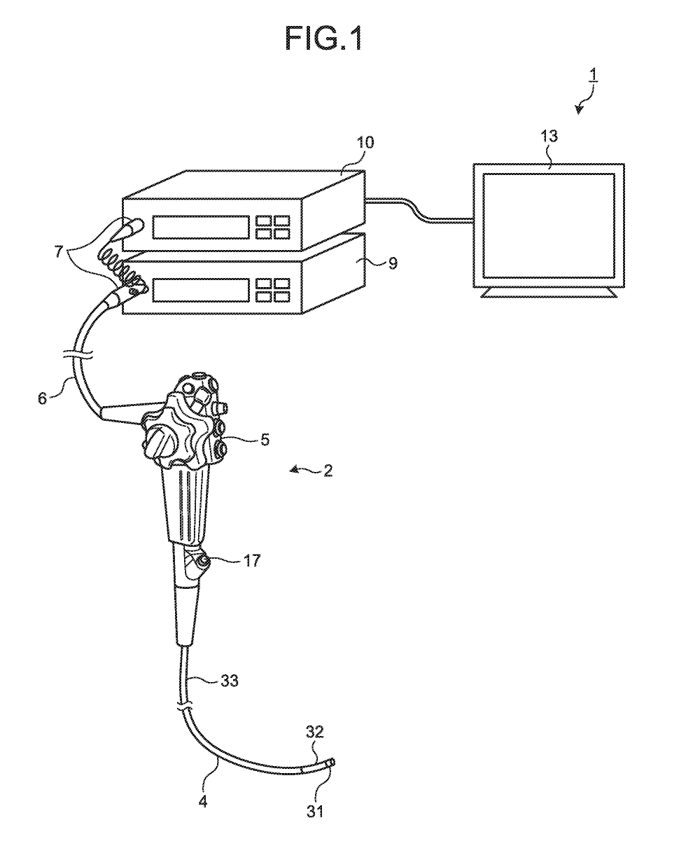

FIG. 1 is a diagram schematically illustrating an overall configuration of an endoscope system according to a first embodiment of the present disclosure. As illustrated in FIG. 1, an endoscope system 1 includes an endoscope 2, a universal cord 6, a connector 7, a light source device 9, a processor (control device) 10, and a display device 13.

The endoscope 2 captures an in-vivo image of a subject and outputs an imaging signal by allowing an insertion portion 4 to be inserted into the subject. A bundle of electric cables inside the universal cord 6 is extended to a distal end of the insertion portion 4 of the endoscope 2 and is connected to an imaging device provided in a distal end portion 31 of the insertion portion 4.

The connector 7 is provided at a proximal end of the universal cord 6, is connected with the light source device 9 and the processor 10, applies predetermined signal processing to the imaging signal output by the imaging device in the distal end portion 31, the imaging device being connected with the universal cord 6 and performs analog-digital conversion (A/D conversion) for the imaging signal, and outputs the imaging signal as an image signal.

The light source device 9 is configured from a white LED, for example. Pulse white light lighted by the light source device 9 becomes illumination light to be irradiated toward an object from the distal end of the insertion portion 4 of the endoscope 2 through the connector 7 and the universal cord 6.

The processor 10 applies predetermined image processing to the image signal output from the connector 7, and controls the entire endoscope system 1. The display device 13 displays the image signal to which the processing has been applied by the processor 10.

An operating unit 5 provided with various buttons and knobs for operating endoscope functions is connected to the insertion portion 4 of the endoscope 2 on a proximal end side. A treatment tool insertion slot 17 for allowing treatment tools, such as forceps, a radio knife, and an inspection probe, to be inserted into a body cavity of the subject is provided in the operating unit 5.

The insertion portion 4 is configured from the distal end portion 31 in which the imaging device is provided, a bend portion 32 provided in a linked manner to the distal end portion 31 on a proximal end side and bendable in a plurality of directions, and a flexible tube portion 33 provided in a linked manner to the bend portion 32 on a proximal end side. A bend tube (not illustrated) in the bend portion 32 is bent by an operation of a bending operation knob provided in the operating unit 5 and is bendable in up and down, and right and left four directions with pulling and relaxing of a bend wire inserted into the insertion portion 4.

In the endoscope 2, a light guide (not illustrated) that transmits the illumination light from the light source device 9 is provided, and an illumination lens (not illustrated) is arranged in an emission end of the illumination light by the light guide. This illumination lens is provided in the distal end portion 31 of the insertion portion 4, and the illumination light is radiated toward the subject.

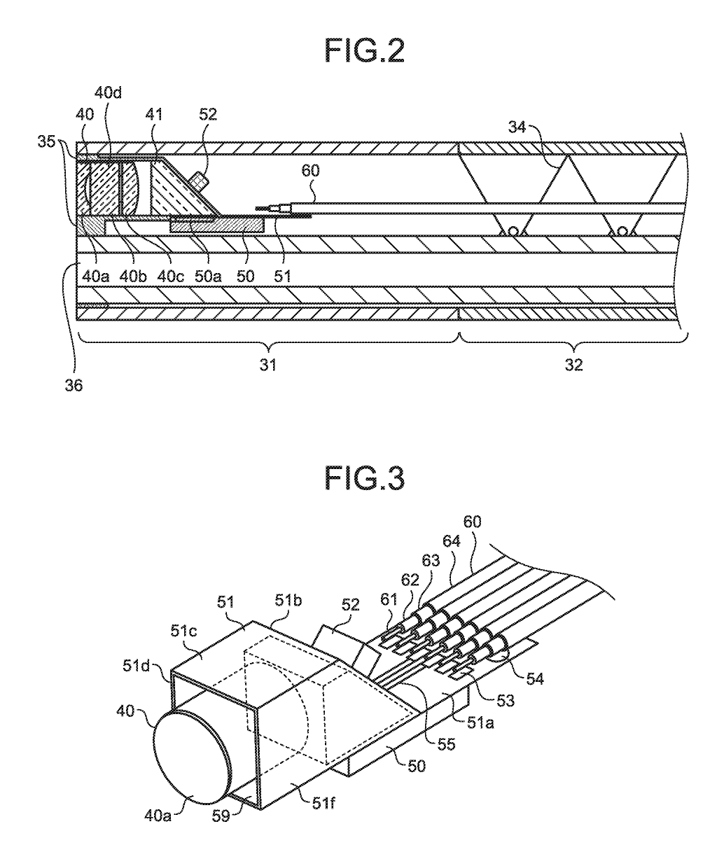

Next, a configuration of the distal end portion 31 of the endoscope 2 will be described in detail. FIG. 2 is a partial sectional view of the distal end of the endoscope 2. FIG. 2 is a sectional view of a case in which the distal end is cut in a surface parallel to an optical axis direction of incident light of the imaging device provided in the distal end portion 31 of the endoscope 2 and including a vertical axis. FIG. 2 illustrates the distal end portion 31 and a part of the bend portion 32, of the insertion portion 4 of the endoscope 2.

As illustrated in FIG. 2, the bend portion 32 is bendable in the up and down, and right and left four directions with pulling and relaxing of the bend wire inserted into a bend tube 34. An imaging device 100 is provided in an upper portion inside the distal end portion 31 running to the distal end side of the bend portion 32, and a treatment tool channel 36 in which various treatment tools are extended is formed in a lower portion inside the distal end portion 31.

The imaging device 100 includes an objective optical system 40 that concentrates incident light, a prism 41 that reflects the light concentrated by the objective optical system 40, and an image sensor 50 that generates an image signal on the basis of the light entering through the prism 41. The imaging device 100 is glued inside the distal end portion 31 with an adhesive. The distal end portion 31 is formed of a hard member for forming an internal space in which the imaging device 100 is accommodated. An outer peripheral portion of the proximal end of the distal end portion 31 is covered with a flexible cover tube (not illustrated). A member positioned on the proximal end side with respect to the distal end portion 31 is configured from a flexible member so that the bend portion 32 is bendable.

The objective optical system 40 includes a plurality of objective lenses 40a, 40b, and 40c, and a lens frame 40d that covers a periphery of the objective lenses 40a, 40b, and 40c. The lens frame 40d and the objective lens 40a are inserted and fixed to a distal end fixing portion 35 inside the distal end portion 31 and thus are fixed to the distal end portion 31. The lens frame 40d used in the first embodiment is formed of a soft material. The plurality of objective lenses 40a, 40b, and 40c are inserted into and are thus held by a cylindrical portion of a light-shielding member including a flexible printed board 51 (hereinafter, referred to as FPC board) described below, in addition to by the lens frame 40d.

The image sensor 50 includes a light-receiving portion 50a that receives light reflected by the prism 41 and performs photoelectric conversion to generate an electrical signal. The image sensor 50 is a landscape-type image sensor arranged in such a manner that a principal plane on which the light-receiving portion 50a is formed becomes horizontal, that is, parallel to the optical axis of the objective optical system 40, and the prism 41 is arranged on and glued to the light-receiving portion 50a. Further, an electrode pad (not illustrated) is formed on a proximal end of the image sensor 50, and the FPC board 51 to which a signal cable 60 is connected is connected to the electrode pad. An electronic component 52 that drives the image sensor 50 and the like are mounted on the FPC board 51. The image sensor 50 in the first embodiment of the present disclosure is a charge coupled device (CCD)-type or complementary metal oxide semiconductor (CMOS)-type semiconductor image sensor.

Proximal ends of the signal cables 60 are extended in a proximal end direction of the insertion portion 4. The bundle of electric cables is inserted and arranged in the insertion portion 4 and is extended up to the connector 7 through the operating unit 5 and the universal cord 6 illustrated in FIG. 1. The signal cable 60 is a coaxial cable, and includes a core wire 61 provided in a central portion and which transmits an electrical signal, an internal insulator 62 formed to cover the core wire 61, an external conductor 63 formed to cover the internal insulator 62, and an external insulator 64 formed to cover the external conductor 63. The core wire 61 is connected to the image sensor 50 through an electrode portion of the FPC board 51, and to which a drive signal to the image sensor 50 is transmitted and which transmits an electrical signal corresponding to an image captured by the image sensor 50 to the processor 10. Further, the external conductor 63 is connected to an external power supply device, and supplies a power supply voltage to the image sensor 50.

The light entering through one end of the objective optical system 40 is concentrated by the objective lenses 40a to 40c and enters the prism 41. The light-receiving portion 50a selected from a CCD image sensor, a CMOS image sensor, and the like is formed at a position where the light-receiving portion 50a may receive the light radiated through the prism 41, and converts the received light into an imaging signal. The imaging signal is output to the processor 10 through the signal cable 60 connected to the FPC board 51 and the connector 7. In the present specification, the side where the light of the objective optical system 40 enters, that is, the side where the objective lenses 40a to 40c are arranged is called front end, and the side where the signal cable 60 is arranged is called rear end.

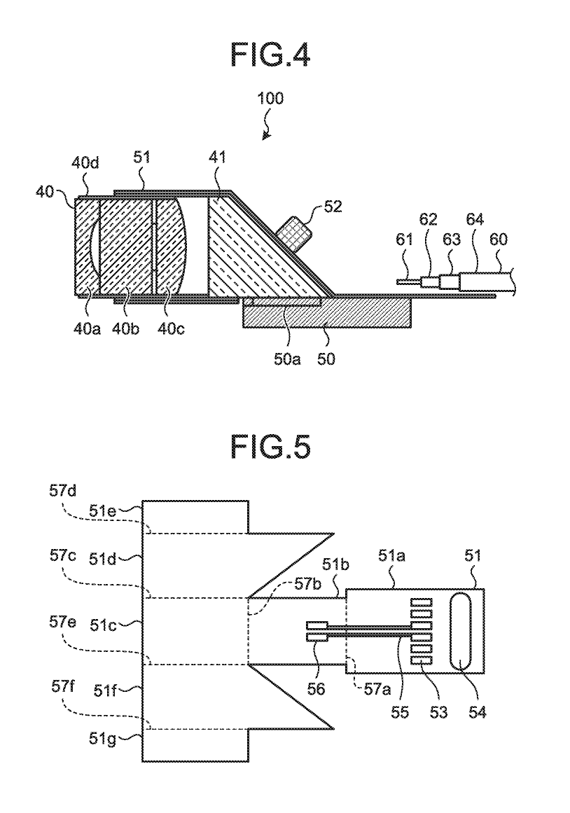

Next, the imaging device 100 will be described with reference to the drawings. FIG. 3 is a perspective view of the imaging device 100 used in the endoscope system 1 according to the first embodiment. FIG. 4 is a sectional view of the imaging device 100 of FIG. 3 (a section of when the imaging device 100 is cut in a surface parallel to the optical axis direction of the incident light of the imaging device 100 and including the vertical axis). FIG. 5 is a development view of the flexible printed board 51 used in the imaging device 100 of FIG. 3.

The imaging device 100 has the FPC board 51 arranged to cover a part of the objective optical system 40 and an outer periphery of the prism 41, as illustrated in FIG. 3. The FPC board 51 used in the first embodiment has a light-shielding layer formed on surfaces coming in contact with the objective optical system 40 and the prism 41 (on a back side of the sheet, of the FPC board 51 illustrated in FIG. 5). As the light-shielding layer, a solder resist, which is used as an insulating layer of the FPC board 51 and is colored in black, may be used. The FPC board 51 functions as a light-shielding member.

As illustrated in FIG. 5, the FPC board 51 includes a surface 51a arranged on a principal plane (where the light-receiving portion 50a is formed) of the image sensor 50, a surface 51b arranged along a reflective surface of the prism 41, and surfaces 51c, 51d, 51e, 51f, and 51g that configure a cylindrical portion 59 that covers side surfaces of the prism 41 and allows the objective optical system 40 to be inserted into. The FPC board 51 forms the cylindrical portion 59 that covers the side surfaces of the prism 41 and allows a part of the objective optical system 40 to be inserted into by being folded at boundaries 57a, 57b, 57c, 57d, 57e, and 57f of the surfaces illustrated by the dotted lines in FIG. 5.

On the surface 51a, core wire connection electrodes 53 to which core wires 61 are respectively connected, an external conductor connection electrode 54 to which the external conductor 63 is connected, and wiring 55 that connects an electrode 56 formed on the surface 51b and the core wire connection electrode 53 are formed. The electronic component 52 is mounted on the electrode 56 formed on the surface 51b. Wiring may be provided on the other surfaces 51c to 51g.

The cylindrical portion 59 is formed to come in contact with the side surfaces of the prism 41 and is formed in such a manner that a section shape of a hollow portion of the cylindrical portion 59 forms approximately the same shape as the shape of the prism 41 as viewed from the front end side. In a case where the side surfaces of the prism 41 are arcs, the section shape of the cylindrical portion 59 may just be formed into a cylindrical shape along the side surfaces (arcs) of the prism 41. An outer diameter of the objective optical system 40 is a size inscribed with a square as an incident surface as the front end of the prism 41, and the cylindrical portion 59 is formed to have a size in which all of inner walls are in contact with a side surface of the objective optical system 40 when the objective optical system 40 is inserted into the cylindrical portion 59. After the objective optical system 40 is inserted into the cylindrical portion 59 and is positioned in the optical axis direction, the objective optical system 40 is glued and fixed to the inner walls of the cylindrical portion 59. A gap between the objective optical system 40 and the cylindrical portion 59 may be sealed with a sealing resin or the like.

In the first embodiment, the cylindrical portion 59 is formed to have the size in which all the inner walls are in contact with the objective optical system 40. Therefore, the positioning of the objective optical system 40 may be easily performed. Further, the space between the objective optical system 40 and the prism 41, and the side surfaces of the prism 41, which stray light or the like may enter, are covered with the thin FPC board 51 having excellent light-shielding properties. Therefore, an influence of light from outside may be decreased while reduction in the diameter is achieved. Further, the light-shielding member is formed of the FPC board 51. Therefore, mounting of the electronic component 52 and routing of the wiring may be performed on the surfaces of the FPC board 51 used as a light-shielding member. Still further, when the wiring 55 is formed on the surface of the FPC board 51 used as a light-shielding member, heat radiation from heat generating portions such as the image sensor 50 and the electronic component 52 may be improved by heat conduction with a metal material used as the wiring material.

In the first embodiment, one sheet of FPC board 51 is folded and used as the light-shielding member. However, separate FPC boards, for example, an FPC board including the surface 51a separated at the boundary 57a and an FPC board including the surfaces 51b, 51c, 51d, 51e, 51f, and 51g may be electrically conducted with a wire or the like. Alternatively, as the board including the surfaces 51b, 51c, 51d, 51e, 51f, and 51g, a rigid flexible board having flexibility may be used. Further, any material other than a board may be used to form the light-shielding member as long as the material has flexibility.

Note that the cylindrical portion 59 may not have the size in which all the inner walls are in contact with the side surface of the objective optical system 40. However, it is favorable to employ a size in which the inner walls positioned right and left of the objective optical system 40 are in contact with the side surface of the objective optical system 40 from a viewpoint of positioning.

### Second Embodiment

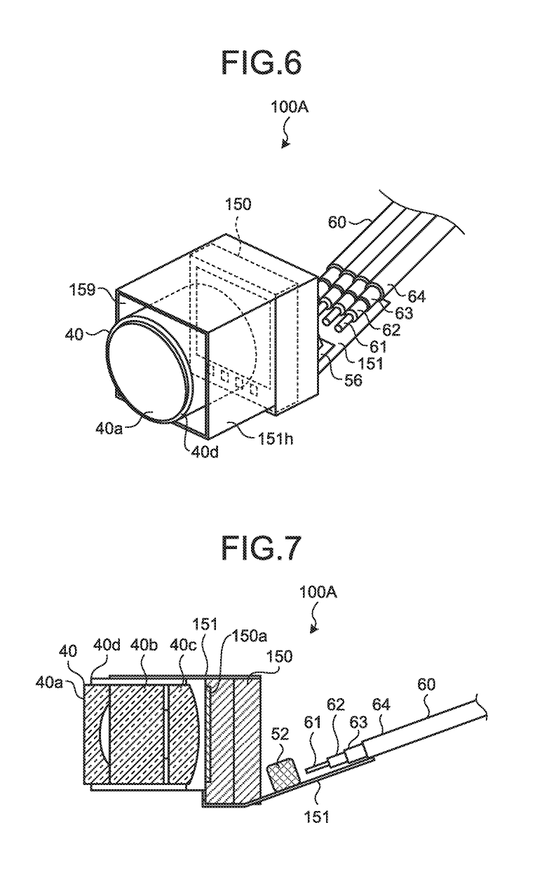

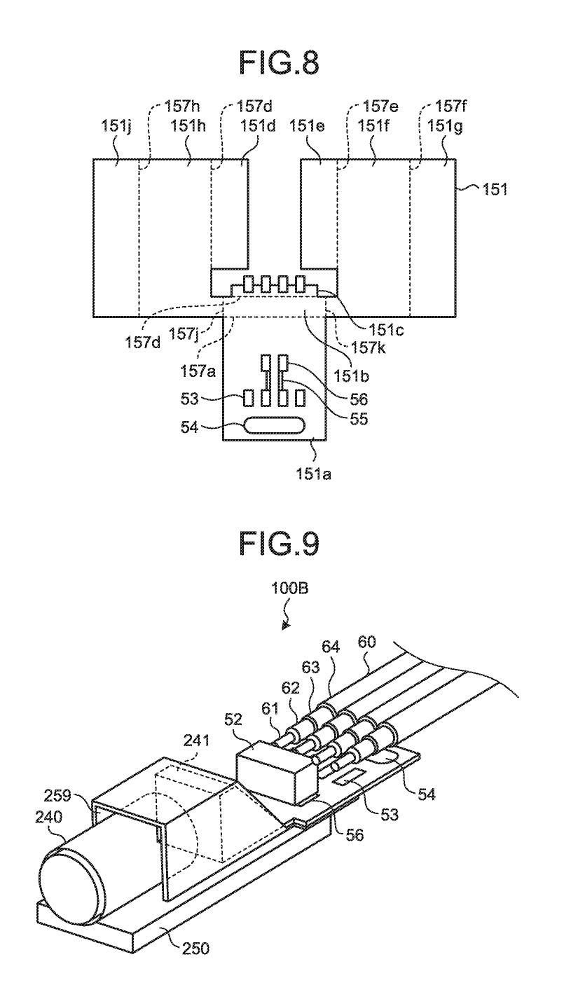

In an imaging device according to a second embodiment, an image sensor is arranged in a portrait manner such that a principal plane on which a light-receiving portion is formed becomes perpendicular to an optical axis of an objective optical system. FIG. 6 is a perspective view of an imaging device according to the second embodiment. FIG. 7 is a sectional view of the imaging device of FIG. 6. FIG. 8 is a development view of a flexible printed board used in the imaging device of FIG. 6.

An imaging device 100A has an FPC board 151 arranged to cover a part of an objective optical system 40 and an outer periphery of an image sensor 150, as illustrated in FIG. 6. The FPC board 151 used in the second embodiment has a light-shielding layer formed on surfaces coming in contact with the objective optical system 40 and the imaging sensor 150 (on a front side of the sheet, of the FPC board 151 illustrated in FIG. 8). As the light-shielding layer, a solder resist, which is used as an insulating layer of the FPC board 151 and is colored in black, may be used. The FPC board 151 functions as a light-shielding member.

As illustrated in FIG. 8, the FPC board 151 includes a surface 151a extending to a rear end side of the image sensor 150, a surface 151b arranged on a bottom surface side, of side surfaces of the image sensor 150, a surface 151c arranged on a principal plane side of the image sensor 150, and surfaces 151d, 151e, 151f, 151g, 151h, and 151j that configure a cylindrical portion 159 that covers side surfaces perpendicular to the principal plane of the image sensor 150 and allows the objective optical system 40 to be inserted into. The FPC board 151 forms the cylindrical portion 159 that covers the side surfaces of the image sensor 150 and allows a part of the objective optical system 40 to be inserted into by being folded at boundaries 157a, 157b, 157d, 157e, 157f, 157h, 157j, and 157k of the surfaces illustrated by the dotted lines in FIG. 8. The surfaces 151d and 151e configure the bottom surface of the cylindrical portion 159, and the surfaces 151g and 151j configure an upper surface of the cylindrical portion 159.

On the surface 151a, core wire connection electrodes 53 to which core wires 61 are respectively connected, an external conductor connection electrode 54 to which an external conductor 63 is connected, an electrode 56 on which an electronic component 52 is mounted, and wiring 55 that connects the electrode 56 and the core wire connection electrode 53 are formed. On the surface 151c, a flying lead 58 connected to an electrode pad of the image sensor 150 is arranged. Wiring may be provided on the other surfaces 151b, and 151d to 151j.

The cylindrical portion 159 is formed to have a size in which all of inner walls are in contact with a side surface of the objective optical system 40. After the objective optical system 40 is inserted into the cylindrical portion 159 and is positioned in the optical axis direction, the objective optical system 40 is fixed and glued to the inner walls of the cylindrical portion 159. A gap between the objective optical system 40 and the cylindrical portion 159 may be sealed with a sealing resin or the like.

In the second embodiment, the cylindrical portion 159 is formed to have the size in which all the inner walls are in contact with the objective optical system 40. Therefore, the positioning of the objective optical system 40 may be easily performed. Further, a space between the objective optical system 40 and the image sensor 150, which stray light or the like may enter, is covered with the thin FPC board 151 having excellent light-shielding properties. Therefore, an influence of light from outside may be decreased while reduction in the diameter is achieved. Further, the light-shielding member is formed of the FPC board 151. Therefore, mounting of the electronic component 52 and routing of the wiring 55 may be performed on the surfaces of the FPC board 151 used as a light-shielding member. Still further, when the wiring is formed on the surface of the FPC board 151 used as a light-shielding member, heat radiation from heat generating portions such as the image sensor 150 and the electronic component 52 may be improved by heat conduction with a metal material used as the wiring material.

Note that the cylindrical portion 159 may not have the size in which all the inner walls are in contact with the side surface of the objective optical system 40. However, it is favorable to employ a size in which the inner walls positioned right and left of the objective optical system 40 are in contact with the side surface of the objective optical system 40 from a viewpoint of positioning.

### Third Embodiment

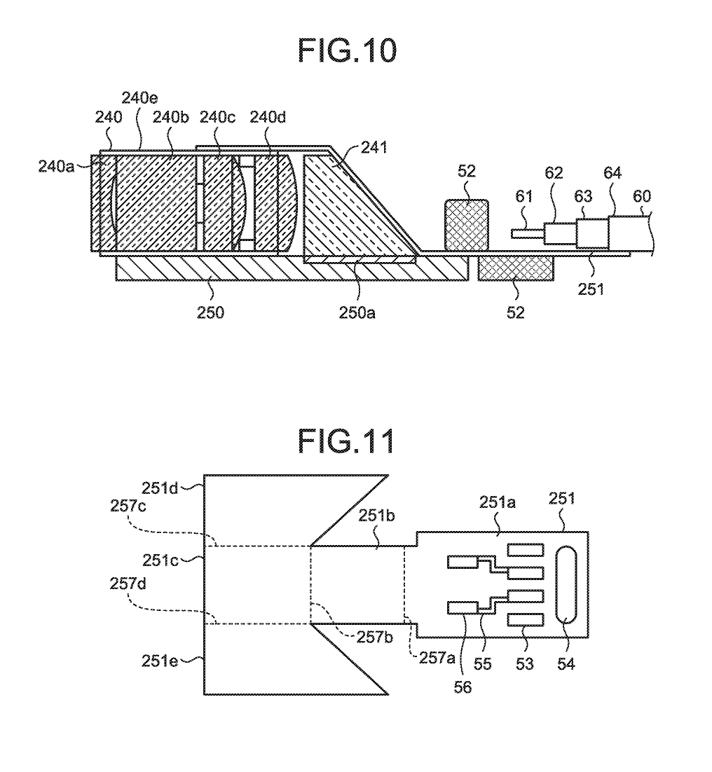

In an imaging device according to a third embodiment, an image sensor has a light-receiving portion arranged in parallel to an optical axis of an objective optical system in a landscape manner, and the objective optical system is arranged on the image sensor. FIG. 9 is a perspective view of an imaging device according to the third embodiment. FIG. 10 is a sectional view of the imaging device of FIG. 9. FIG. 11 is a development view of a flexible printed board used in the imaging device of FIG. 9.

As illustrated in FIGS. 9 and 10, an imaging device 100B has a prism 241 and an objective optical system 240 arranged on an image sensor 250, and an FPC board 251 arranged to cover a part of the objective optical system 240 and an outer periphery of the prism 241. The objective optical system 240 includes a plurality of objective lenses 240a, 240b, 240c, and 240d, and a lens frame 240e that covers a periphery of the objective lenses 240a, 240b, 240c, and 240d. The FPC board 251 used in the third embodiment has a light-shielding layer formed on surfaces coming in contact with the objective optical system 240 and the prism 241 (on a back side of the sheet, of the FPC board 251 illustrated in FIG. 11). As the light-shielding layer, a solder resist, which is used as an insulating layer of the FPC board 251 and is colored in black, may be used. The FPC board 251 functions as a light-shielding member.

As illustrated in FIG. 11, the FPC board 251 includes a surface 251a arranged on a principal plane (where a light-receiving portion 250a is formed) of the image sensor 250, a surface 251b arranged along a reflective surface of the prism 241, and surfaces 251c, 251d, and 251e that cover side surfaces of the prism 241 and cover a part of a side surface of the objective optical system 240. The FPC board 251 covers the side surface of the prism 241 and a part of the side surfaces of the objective optical system 240 by being folded at boundaries 257a, 257b, 257c, and 257d of the surfaces illustrated by the dotted lines in FIG. 11.

On the surface 251a, core wire connection electrodes 53 to which core wires 61 are respectively connected, an external conductor connection electrode 54 to which an external conductor 63 is connected, an electrode 56 on which an electronic component 52 is mounted, and wiring 55 that connects the electrode 56 and the core wire connection electrode 53 are formed. The electronic component 52 may be mounted on the surface 251b, and wiring may be provided on the surfaces 251b to 251e.

A cylindrical portion 259 is configured from the surfaces 251c, 251d, and 251e, and the principal plane of the image sensor 250, and is formed to have the size in which all the inner walls are in contact with the side surface of the objective optical system 240. After the objective optical system 240 is inserted into the cylindrical portion 259 and is positioned in an optical axis direction, the objective optical system 240 is fixed and glued to the cylindrical portion 259, that is, the FPC board 251 and the image sensor 250. A gap between the objective optical system 240 and the cylindrical portion 259 may be sealed with a sealing resin or the like.

In the third embodiment, the cylindrical portion 259 is formed to have the size in which all the inner walls are in contact with the objective optical system 240. Therefore, the positioning of the objective optical system 240 may be easily performed. Further, a space between the objective optical system 240 and the prism 241, and the side surfaces of the prism 241, which stray light or the like may enter, are covered with the thin FPC board 251 having excellent light-shielding properties. Therefore, an influence of light from outside may be decreased while reduction in the diameter is achieved. Further, the light-shielding member is formed of the FPC board 251. Therefore, mounting of the electronic component 52 and routing of the wiring 55 may be performed on the surfaces of the FPC board 251 used as a light-shielding member. Still further, when the wiring is formed on the surface of the FPC board 251 used as a light-shielding member, heat radiation from heat generating portions such as the image sensor 250 and the electronic component 52 may be improved by heat conduction with a metal material used as the wiring material.

Note that the cylindrical portion 259 may not have the size in which all the inner walls are in contact with the side surface of the objective optical system 240. However, it is favorable to employ a size in which the inner walls positioned right and left of the objective optical system 240 are in contact with the side surface of the objective optical system 40 from a viewpoint of positioning. Note that the electronic component 52, and the core wire 61 and the external conductor 63 of the signal cable 60 are electrically connected with the electrode portion of the FPC board 51 (151 or 251) using solder. In the drawings of the above-described embodiments, illustration of the solder and the like used for the connection is omitted.

According to the present disclosure, an imaging device that may be downsized and may shield light entering from between an objective optical system and a prism, from a side surface of the prism, and from between the objective optical system and an image sensor, without using a lens holder or an image sensor holder, may be obtained.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An imaging device comprising:
an optical system configured to concentrate incident light;
an image sensor configured to receive the light entering from the optical system and perform photoelectric conversion to generate an electrical signal;
a flexible printed board on which a signal cable and an electronic component are mounted, the flexible printed board being connected to an electronic pad of the image sensor; and
a light-shielding member arranged between the optical system and the image sensor, a part of the light-shielding member being folded or bent to be in contact with a side surface of the optical system, and the light-shielding member being configured to shield the light entering from a direction of the side surface of the optical system and/or from between the optical system and the image sensor,
wherein the light-shielding member is a board including a circuit layer and electrically conducting with the flexible printed board.

2. The imaging device according to claim 1, wherein
the image sensor is arranged such that a principal plane on which the light receiving portion is perpendicular to an optical axis of the optical system,
the light-shielding member includes a cylindrical portion in a cylindrical shape contacting with a side surface perpendicular to the principal plane of the image sensor, and
at least a part of the optical system is positioned in the cylindrical shape of the light-shielding member.

3. The imaging device according to claim 1, wherein
the optical system includes:
an objective optical system including a plurality of objective lenses; and
a prism arranged on the light receiving portion and which reflects the light concentrated by the objective optical system,
the image sensor is arranged such that a principal plane on which the light receiving portion is formed is parallel to an optical axis of the objective optical system,
the light-shielding member includes a cylindrical portion in a cylindrical shape contacting with a side surface of the prism, and
at least a part of the objective optical system is positioned in the cylindrical shape of the light-shielding member.

4. The imaging device according to claim 1, wherein the optical system includes:
an objective optical system including a plurality of objective lenses; and
a prism arranged on the light receiving portion and which reflects the light concentrated by the objective optical system,
the image sensor is arranged such that a principal plane on which the light receiving portion is parallel to an optical axis of the objective optical system, and the objective optical system is arranged on the image sensor,
the light-shielding member forms a cylindrical portion in a cylindrical shape together with a principal plane of the image sensor, and
the prism and a part of a side surface of the objective optical system are positioned in the cylindrical portion.

5. The imaging device according to claim 3, wherein
the light-shielding member includes the flexible printed board, and
the flexible printed board arranged in parallel to the principal plane of the image sensor is folded along a reflective surface of the prism and is folded or bent to contact with a side surface of the prism.

6. An endoscope system adapted to be inserted into a living body and to capture an inside of the living body, the endoscope system comprising:
an endoscope including the imaging device according to claim 1 in a distal end portion.
